(19) United States
(12) Patent Application Publication
(10) Pub. No.: US 2007/0148188 A1
(43) Pub. Date: Jun. 28, 2007

(54) Anthrax vaccine

(75) Inventors: Michael J. Caulfield, Rahway, NJ (US); James C. Cook, Lansdale, PA (US); Joseph G. Joyce, Lansdale, PA (US)

Correspondence Address: MERCK AND CO., INC, P O BOX 2000, RAHWAY, NJ, 07065-0907, US

(73) Assignee: MERCK & CO., INC., Rahway, NJ (US)

(21) Appl. No.: 10/566,088

(22) PCT Filed: Jul. 30, 2004
(86) PCT No.: PCT/US04/25033

§ 371 Date: Jan. 26, 2006

Related U.S. Application Data
(60) Provisional application No. 60/491,478, filed on Jul. 30, 2003.

(51) Int. Cl. A61K 39/295; A61K 39/116; A61K 39/095; C07K 14/22

(52) U.S. Cl. 424/201.1; 424/203.1; 424/250.1; 530/350

(57) ABSTRACT

This invention provides a conjugate between poly-D-gamma glutamic acid and a carrier protein. The conjugate can be used for therapeutic or prophylactic immunization against anthrax infections. The invention also includes methods of purifying poly-D-gamma glutamic acid, methods of conjugation, vaccines and methods of vaccination against B. anthracis.

### FIELD OF THE INVENTION

The invention relates to the field of vaccination.

### BACKGROUND

Anthrax infection, a disease caused by the spore-forming bacterium Bacillus anthracis, is highly lethal in the pulmonary or inhalation form (For general reviews see Friedlander, A. M., “Anthrax: clinical features, pathogenesis, and potential biological warfare threat”. Current Clinical Topics in Infectious Diseases, 2000; 20:335-49. Little, S. F. and Ivins, B. E., “Molecular Pathogenesis of Bacillus anthracis Infection”. Microbes and Infection Institute Pasteur, 1999; 1:131-9. Mock, M. and Fouet, A., “Anthrax”. Annual Review of Microbiology, 2001; 55:647-7). With an increased awareness of the potential of this pathogen as a weapon, the need for a readily available, safe and effective vaccine for wide public use has grown (See, e.g., Ibrahim et al., 1999). Current vaccine preparations for human use are typically attenuated live spores or cell-free secretion products of B. anthracis adsorbed to alum (AVA). The former preparation was reportedly used in the Soviet Union and had a number of toxicities and production issues. In the US, AVA is reported to be used exclusively. Neither vaccine has the characteristics preferred for broad distribution in the event of an emergency or for general public prophylaxis, i.e., long term protection, high level efficacy with limited number of immunizations, low reactogencity, and reliable, safe production. Thus, there is a need for new vaccines that provide a better fit to these criteria.

B. anthracis has two virulence factors, the tripartite toxin (PA, LF, EF) encoded on the plasmid pX01 and the PGGA capsule, which is encoded on as separate plasmid (pX02). The currently licensed vaccine targets only the toxin component but does not elicit immunity to vegetative B. anthracis bacteria, which are protected from innate immunity by the PGGA capsule. The capsule was not generally considered a viable vaccine candidate since purified capsular material was known to be poorly immunogenic in animals (Hanby W E, Rydon H N. The capsular substance of Bacillus anthracis. Biochem. J. 1946;40:297-309). The capsule was, however, shown to be antigenic since antisera raised by immunization of animals with whole bacteria could bind to the purified capsular material.

The PGGA capsule is present on virulent B. anthracis and on attenuated strains of B. anthracis that express the pX02 plasmid (which encodes the genes required for capsule production) but lack the pX01 plasmid (which encodes the anthrax toxin genes). Currently, both wild type and the pX01-negative strains are classified by the CDC and the USDA as Select Agents as codified in 42 CFR 73. An alternative source for the capsule material may be other Bacillus species such as B. anthracis, which has been reported to express a PGGA capsule similar to that of B. anthracis (Bovarnick M. The formation of extracellular d(−)-glutamic acid polypeptide by Bacillus subtilis. J. Biol. Chem. 1942; 145:415-424). Other Bacillus species have also been reported to produce PGGA including B. megaterium and Bacillus M. (Guex-Holzer S, Tomcsik J. The isolation and chemical nature of capsular and cell-wall haptens in a Bacillus species. J. Gen. Microbiol. 1956; 14:14-25), and B. licheniformis (Gardner J M, Troy F A. Chemistry and biosynthesis of the poly(gamma-D-glutamyl) capsule in Bacillus licheniformis. Activation, racemization, and polymerization of glutamic acid by a membranous polyglutamyl synthetase complex. J Biol Chem 1979;254:6262-9).

One of the current anthrax vaccines is produced from a culture filtrate of germinating B. anthracis spores (Puziss, 1962; Puziss, 1963). The major component of this formulation is PA83 with some LF and EF. No further enrichment or purification of the protective component is reportedly performed. Minor, but highly potent reactogenic substances could also be present. Trace amounts of LF and EF purified from the B. anthracis fermentation could theoretically combine with PA cleaved after administration to yield toxins.

The currently licensed vaccine known as anthrax vaccine adsorbed (AVA) is produced by BIOPORT (Lansing, Mich.) under the name BIOTHRAX. The vaccine is a poorly characterized sterile culture filtrate from an attenuated non-encapsulated strain of Bacillus anthracis that is adsorbed to aluminum hydroxide adjuvant. It contains no dead or live bacteria, and it contains unknown amounts of the anthrax toxin components, protective antigen (PA), lethal factor (LF) and edema factor (EF). The final product contains 1.2 μg/mL aluminum hydroxide, 25 μg/mL benzethonium chloride, and 100 μg/mL formaldehyde as preservatives. Efficacy is based on clinical trials conducted from 1955-1959 with a similar vaccine in which efficacy was 92.5% (lower bound of C.I=65%). The BIOTHRAX label states that since the risk of anthrax infection in the general population is low, routine immunization is not recommended. The safety and efficacy of BIOTHRAX in a post-exposure setting has not been established. The vaccine is recommended for individuals 18-65 years old who are at risk for exposure to anthrax spores. There is no indication for use in pediatric or geriatric populations, and the label states that pregnant women should not be vaccinated against anthrax unless the potential benefits of vaccination clearly outweigh the potential risks to the fetus.

A variety of alternative preparations designed to address the toxicity issues raised above have been reported in the literature. These range from the use of attenuated B. anthracis strains with enhanced PA production, to acellular recombinant protein products to naked DNA preparations.

Recently, conjugates between PGGA or peptides of PGGA and recombinant Protective Antigen or exotoxin were reported by Schneerson, et al., 2003. Poly(gamma-D-glutarnic acid) protein conjugates induce IgG antibodies in mice to the capsule of Bacillus anthtracis: A potential addition to the anthrax vaccine. PNAS 100:8945-8950. The authors reported that while a conjugate consisting of a 10-mer of PGGA bound to a protein gave the best immunogenic response, conjugates made with the natural PGGA were the least effective of their conjugates. Moreover, the authors reported that PGGA-protein conjugates made with natural PGGA formed precipitates during synthesis and were produced in low yields. The authors reported that the conjugates were immunogenic in mice. However, the report did not address whether the antibody response protected the mice from disease or debilitating effects caused by infection with anthrax.

Thus, the problem of efficiently making a soluble, effective vaccine against death, disease, cellular toxicity or the debilitating effects caused by infection with B. anthracis using native PGGA conjugated to a protein carrier appears to remain unanswered.

### SUMMARY OF THE INVENTION

An aspect of the present invention is a protein-polypeptide conjugate, or a pharmaceutically acceptable salt thereof, in which a multitude of high molecular weight poly-D-gamma-glutamic acid polypeptides, each of which comprise extracellular epitopes of the Bacillus anthracis capsular protein, are conjugated to the surface of a carrier protein or protein complex.

In particular embodiments, the polypeptides are conjugated to the protein by covalently joining peptides to reactive sites on the surface of the protein. The resulting structure is a conjugate. A reactive site on the surface of the protein is a site that is chemically active or that can be activated and is sterically accessible for covalent joining with a peptide. A preferred reactive site is the epsilon nitrogen of the amino acid lysine. Covalently joined refers to the presence of a covalent linkage that is stable to hydrolysis under physiological conditions. Preferably, the covalent linkage is stable to other reactions that may occur under physiological conditions including adduct formation, oxidation, and reduction. The covalent joining of peptide to protein is achieved by “means for joining”. Such means cover the corresponding structure, material, or acts described herein and equivalents thereof.

In a particular embodiments of this aspect of the invention, the carrier protein is an antigenic protein useful in the art of vaccination. In a particular embodiment of the invention, the antigenic protein is the outer membrane protein complex (OMPC) of Neiserria meningitidis. In other embodiments, the carrier protein can be tetanus toxoid, diphtheria toxoid, Hepatitis B Surface Antigen (HBsAg), Hepatitis B core antigen (HBcAg), recombinant Protective Antigen or the L1 protein of the Human Papilloma Virus Virus Like Particle type 6, 11 or 16.

In particular embodiments of this invention, the PGGA is purified to above 80%, preferably about 85% and most preferably above 90% or 95%.

In embodiments, the PGGA is fractionated to remove low molecular weight species of the polymer. In particular embodiments, the PGGA is fractionated to be on average greater than approximately 50 kDa, 100 kDa, 200 kDa, 300 kDa or 400 kDa. In other embodiments, the PGGA is fractionated to be between approximately 50 and 100 kDa, 100 and 200 kDa, 200 and 300 kDa or 300 to 400 kDa.

Another aspect of this invention is a method of making a peptide-protein conjugate by covalently linking PGGA polypeptides to reactive sites on the surface of a protein. In embodiments of this invention, the PGGA is conjugated to the carrier protein by activating the PGGA in a manner that does not lead to significant reduction in the size of the polymer and reacting the activated polymer with an activated carrier protein. It is preferred that the means for joining does not lead to significant scission of the PGGA chain.

In particular embodiments, a tetrabutyl ammonium or equivalent salt of PGGA is reacted with the heterobifunctional reagent N-(epsilon-maleimidocaprioic acid)hydrazide in the presence of an appropriate condensing reagent, for example, N,N′-diisopropyl carbodiinide (or equivalent carbodiimide) or 4-(4,6-dimethoxy[1,3,5]triazin-2-yl)-4-methyl-morpholinium chloride (or equivalent triazine reagent) in dimethylformamide, or other compatible non-aqueous solvent, converted to a soluble salt, preferably sodium, and reacted with a thiolated carrier protein, most preferably OMPC.

In further embodiments, the PGGA is conjugated to the carrier protein via a linker moiety. In particular embodiments, the linker is a monogeneric or bigeneric spacer.

In further embodiments, the carrier protein is the outer membrane protein complex (OMPC) of Neiserria meningitidis and the conjugate can have from about 10%, from about 8% to about 12%, from about 5% to 15%, from about 5% to about 20% or from about 5% to about 25% PGGA polypeptides by weight.

An aspect of this invention is a method of manufacturing the conjugate of this invention including the steps of isolating PGGA, purifying the PGGA, separating or fractionating small molecular weight PGGA from high molecular weight PGGA and conjugating the high molecular weight PGGA to a carrier protein.

Another aspect of this invention is a method of making a vaccine by adjuvanting a PGGA-protein conjugate of this invention and formulating the adjuvanted conjugate with a pharmaceutically acceptable carrier. In particular embodiments the method the adjuvant is an aluminum based adjuvant. In other embodiments, the vaccine further comprises a cationic adjuvant, e.g., the QS21 adjuvant.

Another aspect of the present invention is a combination vaccine wherein one of the antigenic components comprises PGGA polypeptides having an extracellular epitope of the capsular protein of B. anthracis conjugated to amino acids on the surface of a carrier protein. In particular embodiments, the combination vaccine comprises antigenic components selected from Haemophilus influenza, hepatitis viruses A, B, or C, epitopes derived from the M2, hemaglutinin and neuraminidase proteins of Influenza virus types A or B, human papilloma virus, measles, mumps, rubella, varicella, rotavirus, Streptococcus pneumonia and Staphylococcus aureus.

An aspect of this invention provides a vaccine against death, disease, cellular toxicity or the debilitating effects caused by infection by B. anthracis. A vaccine of this invention includes an effective amount of a PGGA-protein conjugate. A vaccine of this invention can also include pharmaceutically acceptable excipients.

An aspect of this invention is a method of vaccinating a patient against disease, toxicity or death caused by B. anthracis. A vaccine of this invention is administered to a patient in a manner appropriate for the induction in the patient of an immune response against the capsular PGGA protein of B. anthracis.

The term “effective amount” means sufficient vaccine composition is administered to a patient so that an immune response results. One skilled in the art recognizes that this level may vary.

The term “patient” means a mammal, particularly domesticated livestock including but not limited to dogs, cats, cows, bulls, steers, pigs, horses, sheep, goats, mules, donkeys, etc. Most preferably, a patient is a human.

### BRIEF DESCRIPTIONS OF THE DRAWINGS

FIG. 1. Antibody Response to B. anthracis PGGA capsule-OMPC conjugate vaccine.

FIG. 2. Antibody Response to B. anthracis PGGA capsule-OMPC conjugate vaccine.

FIG. 3. Structures of heterobifunctional cross-linker EMCH, and the condensing reagents DIPC and DMTMM.

FIG. 4. 1D ¹H NMR analysis of activated PGGA at 25° C. Sample was prepared by adding 650 μl of D₂O (with 0.02% succinic acid and 0.01% d6-DSS) to dried product. The NMR was collected on a 600 MHz Varian instrument in 5 mm tubes at a probe temperature of 25° C.

FIG. 5. Schemes for PGGA activation and conjugation of activated polymer (A) with thiolated OMPC (B) to form covalent adduct (C). The squiggly lines in the adduct show the bonds which are cleaved upon acid hydrolysis to generate 6-aminohexanoic acid and dicarboxyethylhomocysteine.

FIG. 6. Coomassie gel of PGGA-OMPC conjugate and controls.

### DETAILED DESCRIPTION OF THE INVENTION

The present invention provides a conjugate between poly-D-gamma-glutarnic acid (PGGA) and a protein carrier. The conjugate of the present invention is useful as a vaccine against disease, death or debilitation caused by infection by Bacillus anthracis. In preferred embodiments, the protein carrier is OMPC.

A most important aspect of the current invention is the demonstration that native, high molecular weight PGGA capsule can be conjugated to a carrier protein, preferably OMPC, and rendered highly immunogenic. PGGA is a labile polypeptide polymer that readily degrades into smaller fragments when subjected to many common chemical activation procedures. The exemplified conjugation technique demonstrates that PGGA can be activated and conjugated to a carrier protein in a manner that prevents or minimizes degradation of the polymer. We prefer to maintain the PGGA as larger fragments, above 50 kDa, preferably above 100 kDa, 200 kDa and most preferably above 300 kDa for conjugation to the carrier protein. Without wishing to be bound to any particular theory, the high immunogenicity demonstrated by the present conjugates could be due to the high molecular weight of the PGGA after activation.

As taught herein, the PGGA conjugate vaccine of the present invention induced at least 1000-fold higher antibody titers than did the unconjugated PGGA, and protected 100% of mice from death resulting from challenge with live B. anthracis . Immunization with PGGA alone protected only ˜30% (3/10) of challenged mice from death. Immunization with the unconjugated OMPC carrier (formulated on aluminum hydroxyphosphate) (5/9 dead) appeared to have some benefit relative to the unvaccinated control group (5/5 dead). This may be due to the potent immunostimulatory effects of OMPC (Perez-Melgosa M, Ochs H D, Linsley P S, Laman J D, van Meurs M, Flavell R A, Ernst R K, Miller S I, Wilson C B. Carrier-mediated enhancement of cognate T cell help: the basis for enhanced immunogenicity of meningococcal outer membrane protein polysaccharide conjugate vaccine. Eur J Immunol 2001; 31:2373-81) that may have resulted in the activation of phagocytic cells that could kill the challenge bacteria in the absence of capsule-specific antibody. This nonspecific effect of OMPC may have been revealed because of the short time interval (2 weeks) between the final immunization and the challenge with live bacteria. A longer interval between immunization and challenge would probably result in a decline in this activity. Nevertheless, induction of nonspecific immunity by the OMPC carrier could be an advantage of the vaccine when used in a post-exposure setting.

The present invention includes a method for preparing highly purified poly-D-gamma-glutamic acid, “POGA” from extracts of cultured Bacilli, particularly from Bacillus anthracis. This method removes impurities present in crude extracts and increases the purity of the polymer preparation from <70% to >80%, >85%, >90% or >95%. Impurities can include nucleic acids, bacterial proteins, cell wall components, culture medium components, and cell membrane components. Various steps known in the art can be taken to remove these contaminants, e.g., nuclease digestion, pelleting, etc.

Removal of impurities is preferable for developing chemical conjugation methods for coupling the PGGA capsule polymer to immunologic carrier proteins. Impurities could potentially compete for cross-linking agents, thus altering the outcome of chemical reactions. Contaminants could possibly become conjugated to the carrier protein. Therefore, purified PGGA is preferred for the manufacture of conjugates for use in a vaccine. Removal of impurities is also important because they might cause immune interference, thus highly purified immunogen is preferred for the proper design and interpretation of immunogenicity studies.

Partially purified capsule can be obtained by various methods known in the art including precipitation from culture fluid or autoclaving cultures, pelleting and washing the cell wall material. However, it is believed that partially purified extracts of B. anthracis PGGA capsule polymer are too impure to use for manufacturing a vaccine or conducting proper immunogenicity studies. In addition, the presence of impurities, which could vary from lot-to-lot, can complicate attempts to develop reproducible conjugation methods. Thus, the present invention addresses these problems.

Advantageous attributes of the present method include scalability, sanitary processing, selectivity, efficiency and production PGGA in high yields. The unit operations can be scaled-up as desired to produce large quantities of purified PGGA. The process can be carried about in sanitary conditions. Equipment commonly used for the operations can be sanitized to produce purified PGGA under cGMP guidelines, if desired. The method is selective and extremely effective in removing impurities while producing highly purified PGGA. Finally, the method is efficient and produces high yields. The method provides purification without significant loss of PGGA. One can expect step-yields in excess of 90%.

The method will now be described in general. However, skilled artisans will be aware of the routine modifications that can be made. Starting with partially purified extract of B. anthracis PGGA, preferable as a lyophilized solid, one dissolves the material in an appropriate aqueous solvent, preferably water, at approximately 2 mg/ml. The solution is mixed with an extraction solution by adding a solution of 0.004M sodium phosphate, pH 7.0+1M NaCl (Buffer A) or an equivalent buffer. The mixture is loaded onto an ionic fractionation column, preferably a hydroxyapatite chromatography column and washed with Buffer A or an equivalent buffer to remove non-bound material. The PGGA is then eluted with a linear gradient from 0 to 100% 0.4M sodium phosphate, pH 7.0+1M NaCl (Buffer B) or an equivalent buffer. Fractions containing purified PGGA are pooled and concentrated by diafiltration against an aqueous solvent, preferably water, by ultrafiltration. The purified PGGA can then be reduced to a powdered form by commonly use techniques including shell-freezing the ultrafiltered PGGA and lyophilization to dryness. It is preferred that the lyophilized PGGA be stored over desiccant at −70° C.

The present invention provides a method for conjugating purified poly-gamma-D-glutamic acid (PGGA) capsule of B. anthracis to carrier proteins. The method is demonstrated herein by covalently conjugating to the outer membrane protein complex (OMPC) of N. meningitidis to yield a vaccine effective in animal immunogenicity and challenge studies. The strategy for conjugation involves activation of PGGA on a portion of its carboxylic acid side chains with the concomitant introduction of a thiol-reactive group such as a maleimide group or equivalent. The activated PGGA is then reacted with an activated sulfhydryl-containing carrier protein. In the Examples below the carrier protein is thiolated OMPC. The resulting conjugate exemplified herein to be covalently coupled and contains approximately 10% by weight PGGA polymer relative to carrier. The activation level of PGGA can be controlled to within 8-15% of total available reactive carboxyls. Importantly, the conjugate is water-soluble.

One choice for activation of the PGGA polymer is through the alpha-side chain carboxyl groups. Although the PGGA contained a terminal free amino group, it is preferable to avoid a single-point attachment for two reasons: (1) given the high Mw of the polymer, the efficiency of coupling through a single amino group would be very low, and (2) if the resulting amide linkage would be unstable, the polymer chain would be lost upon cleavage.

The most common strategy for derivatization of carboxylic acids involves formation of amide bonds with a nucleophile such as a primary amine or hydrazide. In order for this reaction to proceed, the carboxyl group is first converted to a-reactive carbonyl intermediate by a variety of reagents, including carbodiimides, carbonyl diimidazole, and triazine reagents. Upon subsequent reaction with a nucleophile, a stable, covalent amide linkage is formed. While this approach alone would be expected to form covalent conjugates with a carrier protein in a one-pot reaction, the chemistry and the extent of derivatization would be uncontrolled. Therefore a multi-step approach was used in which PGGA was activated by introduction of a heterobifunctional molecule containing a nucleophile at one end and a thiol-reactive maleimide group at the other end. After reaction and purification, maleimide-activated PGGA (maPGGA) could then be reacted with OMPC which had been chemically derivatized to introduce free sulfhydryl groups on a portion of its surface lysines. The procedure to activate carrier proteins in this manner is commonly known in the art.

A number of strategies were attempted to develop a reproducible activation chemistry which did not chemically or physically alter the PGGA polymer other than in the intended manner. One approach was to attempt activation under aqueous conditions since this was the most straightforward and most readily amenable to process scale-up. Previous literature reports had documented that activation of high Mw PGGAs using the water-soluble carbodiimide 1-ethyl-3-(3-dimethylaminopropyl) carbodiimide (EDC) at acidic pH (4.5) resulted in significant mass loss (King, E. C., Watkins, W. J., Blacker, A. J., and Bugg, T. D. H. (1998) J. Polymer Sci. 36, 1995-9). Therefore, an attempt was made to conduct the reaction at near-neutral pH (7.2), but similar results in which the PGGA Mw was reduced from 433,000 to 33,000 were observed.

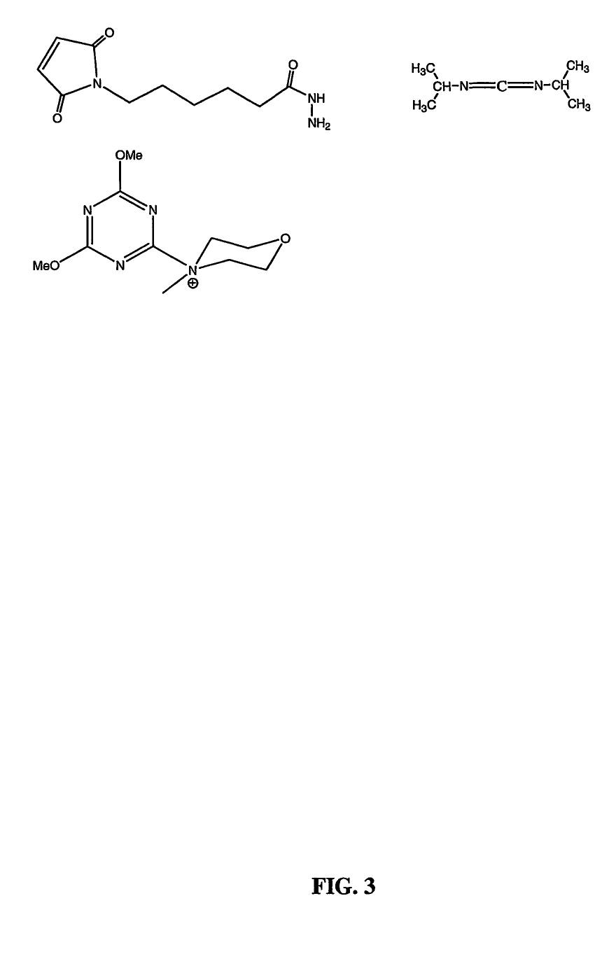

It was then determined whether activation under non-aqueous conditions would prevent the unknown side reactions which resulted in chain scission. Reactions were modeled with poly-alpha-D-glutamic acid (PAGA) because it is commercially available and reacts similarly to PGGA. Both PAGA and PGGA were purified as the Na+ salt which was not directly soluble in any of the organic solvents that were evaluated. However, when the sodium counter ions were replaced by either hydrogen (H+) or tetrabutylammonium (TBA+) and the polymer lyophilized from water, it was readily soluble at 5-10 mg/mL (w/v) in dimethylformamide (DMF). To effect activation, PAGA in DMF was mixed with the heterobifunctional reagent N-(epsilon-maleimidocaproic acid)hydrazide (EMCH) and then an appropriate condensing reagent was added. The condensing reagents which provided the best results were N,N′-diisopropyl carbodiimide (DIPC) and 4-(4,6-dimethoxy[1,3,5]triazin-2-yl)-4-methyl-morpholinium chloride (DMTMM). The structures of all reagents are shown in FIG. 3. Typical reaction conditions for both reagents were 1 hr on ice followed by 3-20 hr at ambient temperature in the dark under nitrogen. Initial attempts to desalt the reaction by gel permeation chromatography in DMF followed by drying and direct resuspension in water were unsuccessful as the TBA+ salts were no longer water soluble. An alternative approach was to dilute the reaction mixture 5-fold with water and dialyze against 1M NaCl. This effected counter ion exchange into the Na+ form after which the product could be dialyzed into water and either dried or stored frozen to preserve maleimide activity.

The extent of product derivatization was determined by NMR analysis where the molar percent of carboxyl groups derivatized was reported as percent side chain loading (% SCL). Signals for both the maleimide group and the methylene protons of the caproic acid portion of the cross-linker were used to quantify the activation levels. It was observed that the activation level was related to both reaction time and the molar charge ratios of DMTMM and EMCH relative to the polymer carboxylic acid repeat unit (RU).

These analyses demonstrated that while DIPC gave incorporation of EMCH, additional peaks in the spectrum corresponding to isopropyl protons indicated that the activating reagent was incompletely removed. The addition of 1-hydroxybenzotriazole (HOBt) or 1-hydroxy-7-azabenzotriazole (HOAt) which are commonly utilized in peptide synthesis to increase reaction yields did not reduce the amount of incorporated DIPC. By comparison, the spectrum of the reaction product generated using DMTMM was very clean, showing exclusively EMCH incorporation. Based on these results, it is preferred that one utilize the DMTMM-based chemistry.

Analytical data summarizing process development is included in Table 1. Since DMTMM was reported to be useful for aqueous-based amide formation 6 we tried performing the reaction in HEPES-buffered saline, pH 7.3 at ambient temperature for 6 hours, using both EMCH and an alternative primary amine-containing cross-linker, 5-(aminopentyl)maleimide (APM). As the data shows, the Mw reduction was still observed, despite the fact that the DMTMM:COOH ratio was reduced to 0.5 from the ratio of 1.0 used for non-aqueous conditions. Reducing the reaction time did not reduce the observed mass reduction. In addition to size reduction, NMR analysis revealed that almost total loss of maleimide functionality was observed in aqueous medium. Signals attributed to the caproic acid methylene protons were present, indicating that the crosslinker was being covalently incorporated, but very little signal was observed for the maleimide ring.

TABLE 1
Analysis summary for PGGA activation studies.

| Reaction¹ | Solvent | % SCL² | Mw (Da)³ |
|---|---|---|---|
| native PGGA | Water | NA | 457,200 |
| DMTMM:COOH 1:1, | DMF | 26 (maleimide) | 220,500 |
| EMCH 16 hr. | | NR (methylene) | |
| DMTMM:COOH 1:1, | DMF | 14 (maleimide) | 286,300 |
| EMCH 3 hr. | | 17 (methylene) | |
| DMTMM:COOH 0.5:1, | HEPES- | 0.3 (maleimide) | 5,122 |
| 5APM, | buffered saline | 27 (methylene) | |
| 6 hr. | | | |
| DMTMM:COOH 0.5:1, | HEPES- | 0.4 (maleimide) | 11,590 |
| EMCH, 6 hr. | buffered saline | 20 (methylene) | |
| native PGGA | water | NA | 621,800 |
| DMTMM:COOH 0.5:1, | HEPES- | 0.3 (maleimide) | 24,520 |
| EMCH, 3 hr. | buffered saline | 5.4 (methylene) | |
| DMTMM:COOH 0.5:1, | HEPES- | 0.5 (maleimide) | 37,970 |
| 5APM, 3 hr. | buffered saline | 5.1 (methylene) | |
| DMTMM:COOH 1:1, | DMF | 20.6 (maleimide) | 495,600 |
| EMCH, | | 23.8 (methylene) | |
| 3 hr. | | | |
| DMTMM:COOH 0.2:1, | DMF | 7.5 (maleimide) | 663,000 |
| EMCH, 3 hr. | | 8.5 (methylene) | |

¹Reaction conditions list the molar charging ratio of DMTMM to COOH, the heterobifunctional reagent employed, and the total reaction time. Native PGGA preparations show starting Mw of untreated polymer. NA, not applicable.
²SCL was determined by quantitation of peaks specific for either maleimide ring protons or methylene protons derived from the crosslinker with those of the glutamic acid backbone. the value is in molar percent.
³Derived from HPSEC/MALLS analysis.

Although maleimides are known to undergo a slow ring-opening hydrolysis in aqueous solution which becomes more pronounced at basic pH, there was no evidence for this form in the spectrum. The possibility existed that maleimide destruction and scission were occurring during the drying step in preparation for NMR analysis. However, when the reaction was repeated and the dialyzed product was analyzed without drying, similar mass reduction was observed while a chemical thiol consumption assay showed no presence of the maleimide group. The aqueous activation route was not further pursued.

Under non-aqueous conditions, the extent of derivatization could be varied by altering the molar charge ratios of both DMTMM and EMCH relative to the RU carboxyl groups as well as by changing the total reaction time. The data indicated that even in the absence of water some mass reduction was observed and this correlated with the degree of polymer modification. However, the resultant polymer sizes were thought to be acceptable based on typical Mw values observed for activated polysaccharide conjugate vaccines. Furthermore, the very close agreement observed between SCL as determined by maleimide and methylene protons indicated that essentially all activated carboxyl groups were derivatized.

Using the conjugate of the present invention, one can produce a vaccine useful for inducing a protective immunity to anthrax. The vaccine can protect a patient from disease, cellular toxicity, death or debilitation caused by infection with B anthracis. The induced immune response can protect the patient from exposure to vegetative bacteria or spores from natural sources or from genetically modified B. anthracis. An advantage of a PGGA based vaccine is that the protective immune response would not be circumvented by genetically modified B. anthracis strains produced for biowarfare or bioterorrism that may contain antibiotic resistance genes. Further, the vaccine of the current invention elicits the rapid production of antibodies against B. anthracis. Therefore, the vaccine could potentially be used in a post-exposure setting to immunize patients suspected of being in contact with B. anthracis spores.

Formulations

The vaccine of the present invention can be formulated according to methods known and used in the art. Guidelines for pharmaceutical administration in general are provided in, for example, Modern Vaccinology, Ed. Kurstak, Plenum Med. Co. 1994; Remington's Pharmaceutical Sciences 18th Edition, Ed. Gennaro, Mack Publishing, 1990; and Modern Pharmaceutics 2nd Edition, Eds. Banker and Rhodes, Marcel Dekicer, Inc., 1990.

The conjugates of the present invention can be prepared as acidic or basic salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

It is preferred that the adjuvant is chosen as appropriate for use with the particular carrier protein used as well as the ionic composition of the final formulation. Consideration should also be given to whether the conjugate alone will be formulated into a vaccine or whether the conjugate will be formulated into a combination vaccine. In the latter instance one should consider the buffers, adjuvants and other formulation components that will be present in the final combination vaccine.

Aluminum based adjuvants are commonly used in the art and include aluminum phosphate, aluminum hydroxide, aluminum hydroxy-phosphate and aluminum hydroxy-phosphate-sulfate. Trade names of adjuvants in common use include ADJUPHOS, MERCK ALlUM and ALHYDROGEL. The conjugate can be bound to or co-precipitated with the adjuvant as desired and as appropriate for the particular adjuvant used.

Non-aluminum adjuvants can also be used if approved for use in the expected patient population. Non-aluminum adjuvants include QS21, Lipid-A and derivatives or variants thereof, Freund's complete or incomplete adjuvant, neutral liposomes, liposomes containing vaccine, microparticles and cytokines or chemokines.

It is preferred that the vaccine be formulated with an aluminum adjuvant. In other preferred embodiments, the vaccine is formulated with both an aluminum adjuvant and QS21.

The conjugate of the present invention can be formulated with other antigens derived from B. anthracis including Protective Antigen, Lethal Factor and Edema Factor or with BIOTHRAX, the currently licensed vaccine for anthrax (BIOPORT, Lansing, Mich.). It is also preferred, in certain embodiments, to formulate the conjugate with immunogens from Haemophilus influenza, hepatitis viruses A, B, or C, human papilloma virus, measles, mumps, rubella, varicella, influenza virus, polio virus, smallpox, rotavirus, Streptococcus pneumoniae and Staphylococcus aureus. Combination vaccines have the advantages of increased patient comfort and lower costs of administration due to the fewer inoculations required.

When formulating combination vaccines one should be mindful of the various buffers and adjuvants used with the other immunogens. Some buffers may be appropriate for some immunogen-adjuvant pairs and not appropriate for others. In particular, one should assess the effects of phosphate levels on the various immunogen-adjuvant pairs to assure compatibility in the final formulation.

Vaccination

The vaccine of the present invention can be administered to a patient by different routes such as intravenous, intraperitoneal, subcutaneous, intranasal or intramuscular. A preferred route is intramuscular. Suitable dosing regimens are preferably determined taking into account factors well known in the art including age, weight, sex and medical condition of the subject; the route of administration; the desired effect; and the particular conjugate and formulation employed. The vaccine can be used in multi-dose vaccination formats. It is expected that a dose would consist of the range of 0.01 μg to 1.0 mg total protein. In embodiments of the present invention the range is 0.1 μg to 100 μg. However, one may prefer to adjust dosage based on the amount of PGGA delivered. In either case these ranges are guidelines. More precise dosages should be determined by assessing the immunogenicity of the conjugate used so that an immunologically effective dose is delivered. An immunologically effective dose is one that stimulates the immune system of the patient to establish a level immunological memory sufficient to provide long term protection against disease, cellular toxicity, debilitation or death caused by infection with B. anthracis . The conjugate is preferably formulated with an adjuvant.

The timing of doses depend upon factors well known in the art. After the initial administration, one or more booster doses may subsequently be administered to maintain antibody titers or immunologic memory. An example of a dosing regime would be day 1, 1 month, a third dose at either 4, 6 or 12 months, and additional booster doses at distant times as needed. Other dosing regimens could consist of fewer doses or a single dose.

A patient or subject, as used herein, is a mammal, particularly domesticated livestock and animals including but not limited to dogs, cats, cows, bulls, steers, pigs, horses, sheep, goats, mules, donkeys, etc. Most preferably a patient is a human. A patient can be of any age at which the patient is able to respond to inoculation with the present vaccine by generating an immune response. The immune response so generated can be completely or partially protective against disease, cellular toxicity, debilitation or death caused by infection with B. anthracis.

The following examples are offered by way of illustration and are not intended to limit the invention in any manner.

### EXAMPLE 1

Purification of PGGA

Starting with partially purified extract of B. anthracis containing PGGA, preferably as a lyophilized solid, one performs the following steps.

1. Dissolve partially purified extract in water to 2 mg/ml.

2. Mix extract solution with Buffer A (0.004M sodium phosphate, pH 7.0+1M NaCl).

3. Load mixture on hydroxyapatite chromatography column and wash out non-bound material with Buffer A.

4. Elute PGGA with a linear gradient from 0 to 100% Buffer B (0.4M sodium phosphate, pH 7.0+1M NaCl).

5. Pool fractions containing pure PGGA (“HA Product”).

6. Concentrate and diafilter HA Product with water by tangential-flow ultrafiltration filtration.

7. Shell-freeze the Ultrafiltration Product and lyophilize to dryness.

8. Store the Lyophilized Product over desiccant at −70° C.

### EXAMPLE 2

Analysis of PGGA

Molecular mass (Mw) determination for four batches of PGGA was performed by HPSEC coupled with multi-angle laser light scattering (MALLS) detection. The analytical system consisted of an AGILENT (Palo Alto, Calif.) 1100 series LC chromatography system and DAWN EOS 18 angle light scattering detector with quasi-elastic light scattering and OPTILAB DSP options (WYATT, Santa Barbara, Calif.). Chromatography was performed at 0.5 mL/min using two ULTRAHYDROGEL Linear 30 cm columns in series behind an ULTRAHYDROGEL guard column (WATERS, Milford, Mass.). Running buffer was 50 mM sodium phosphate, pH 7.2, 0.15 M sodium chloride containing 8 ppm PROCLIN 150 as a preservative. Columns and detectors were maintained at 35° C. For determination of absolute Mw by light scattering, the refractive index increment (dn/dc) of the compound was needed. For proteins this value is typically 0.186 (Wen, J., and Arakawa, T. (2000) Aial. Biocizem. 280, 327-329), but we are not aware of literature data for poly-D-glutamic acids. Since the empirical determination of this value consumed a significant amount of material, it was decided to determine the value for commercially available PAGA and use this result for the PGGA preparations. The empirically-determined dn/dc for PAGA was found to be 0.150+0.002.

The chromatographic behavior of batches of PGGA can vary. It is believed that some factor apart from mass can having an effect on the chromatography. Table 2 gives the various biophysical parameters determined from the MALLS data. The concentrations determined using PAGA dn/dc are relatively close to the concentrations based on dry weight, taking into consideration variation due to moisture content and impurities, and they agree well with independent NMR determinations. Changes in the refractive index increment would result in changes in the estimated Mw and concentrations; however the determinations for the four lots would still be accurate relative to one another. Plots of the RMS radius versus Mw are indicative of the structure of the molecule in solution. (Harding, S. E., Sattelle, D. B., and Bloomfield, V. A., eds. (1992) Laser Light Scattering in Biochemistry, 209-224.) The slopes of the lines for three batches of PGGA fall in the range of 0.62 to 0.67 and suggest an extended random coil conformation. The relationship of the RMS radius to the hydrodynamic radius in each case also suggests that the molecules are present in a random coil configuration. This finding is consistent with various literature reports of poly glutamic acids at neutral pH. (Greenfield, N. and Fasman, G. D. (1969) Biochemistry. 8, 41084116; Arunkumar, A. I., Kumar, T. K. S., and Yu, C. (1997) Biochim. Biophys. Acta. 1338, 69-76; Kimura, T., Takahashi, S., Akiyama, S., Uzawa, T., Ishimori, K., and Morishima, I. (2002) J. Am. Chem. Soc. 124, 11596-11597.)

TABLE 2
Biophysical parameters of batches of PGGA as determined by
HPSEC/MALLS analysis.

| | Batch | | | |
|---|---|---|---|---|
| | 1 | 2 | 3 | 4 |
| M_{w} | 520,500 | 706,600 | 428,500 | 964,000 |
| M_{w}/Mn | 1.194 | 1.285 | 1.286 | 1.044 |
| Rw (nm) | 48.3 | 66.6 | 48.1 | 58.9 |
| Rh_{(w)} (nm) | 25.5 | 30.9 | 26.1 | 35.8 |
| Conc. (mg/ml) | 0.67 | 0.68 | 0.82 | 0.88 |

NMR analyses were performed on a 600 MHz VARLAN (Palo Alto, Calif.) NMR instrument. The PGGA powder was weighed and dissolved in fixed volume of D₂O (99.999%). The D₂O (99.999%) contains 0.01% DMSO of known concentration for the quantitation of polypeptide concentration and its purity. The spectral chemical shift was internally referenced with 0.02% d6-DSS. The acquisition was carried out in 5 mm tubes at a probe temperature of 25° C.

Contaminants associated with the crude lots were not exhaustively profiled by NMR in order to fully identify them, but they were suspected to potentially consist of protein, peptides, or nucleic acids based on their chemical shifts and broadness of the bands. In contrast, the residual impurities associated with purified some batches were all low molecular weight contaminants, primarily glycerol which may have remained as carry-over from the membrane diafiltration steps used to desalt the hydroxyapatite product.

The unambiguous determination of the structure of the polymer as PGGA was performed by 2-dimensional HMBC analysis (Bax, A. Summers, M. F. (1986) J. Am. Clem. Soc. 108, 2093-2094) and analysis of the 2D spectrum with peak assignments. The linkage of PGGA through the gamma-carboxylic group is verified with the presence of a cross peak on HMBC indicating the covalent correlation of the preceding gamma-carboxylic carbon with H-alpha through an amide bond.

Three methods were employed to detect and characterize levels of contaminating protein: (1) Lowry analysis, (2) SDS-PAGE, and (3) amino acid analysis (AAA).

Colorimetric protein determination was performed using a modified form of the Lowry assay (Markwell, M. A., Haas, S. M., Bieber, L. L., Tolbert, N. E. (1978) Anal. Biochem. 87, 206-210) which is less sensitive to non-protein contaminants since it involves a trichloroacetic acid/deoxycholate precipitation step prior to alkaline dissolution of the pellet. In the purification protocol used, PGGA is not precipitated by treatment with 10% TCA, and so any protein detected in the modified Lowry would be expected to result from contaminants. Protein standard was bovine serum albumin (7% solution, NIST standard) and diluent was sterile water. Each batch was assayed at two dilutions and duplicates of each standard or unknown were run. Standard data was fit to a 4-parameter logistic fit curve and unknown concentrations calculated from the curve equation. Results are presented in Table 3. In both cases, the purified lots were below the level of the lowest standard at the lowest dilution assayed. There was detectable, quantifiable protein present for the two crude lots, but it was a minor component of the starting material on a weight percent basis.

TABLE 3
Lowry protein analysis of PGGA batches.

| Lot | [protein] (mg/mL) | Protein (weight %)^{a} |
|---|---|---|
| 1 | 0.032 | 4.8 |
| 2 | 0.026 | 3.8 |
| 3 | <0.019 | <2.3 |
| 4 | <0.019 | <2.2 |

^{a}Based on PGGA concentrations determined by RI from Table 2.

Polyacrylamide gel electrophoresis was performed under denaturing conditions (SDS-PAGE) using standard pre-cast gels and buffer systems (INVITROGEN, Carlsbad, Calif.). Samples were prepared by mixing 1:1 the stock PGGA lots with 2× sample buffer containing 200 mM dithiothreitol as reductant. Samples were incubated at 100° C. for 15 min and then applied to 4-20% Tris-glycine SDS gradient gels of 1 mm thickness. Gels were run at 30 mA/gel constant current for 1 hr. A commercial colloidal Coomassie stain (PRO-BLUE, OWL SYATEMS INC., Portsmouth, N.H.) was used for visualization of protein bands. Densitometry was performed on a KODAK (Rochester N.Y.) IMAGSTATION 1000 imager with associated KODAK 1D software. Visual examination of the gels revealed no bands in any of the lanes. This was consistent with previous data that the PGGA does not stain with Coomassie reagents. Using sensitive parameters, some “bands” were identified by densitometry, but similar ones also were reported in blank lanes of the gel, so it is assumed that these were simply background noise.

Quantitative amino acid analysis was performed using the WATERS (Milford, Mass.) ACCUTAG system. PGGA samples were diluted to 0.5 mg/mL in water, based on the estimated 1.0 mg/mL concentration, and then 20 microliters was transferred to three replicate tubes for hydrolysis. The samples were hydrolyzed in constant boiling 6 N HCl containing 2% phenol at 110° C. for either 20 or 70 hrS. and reconstituted in 40 microliters of 20 mM HCl. Derivatization was performed by adding 120 microliters of WATERS (Milford, Mass.) ACCOFLUOR™ Borate buffer and 40 microliters of reconstituted Waters AccQFluor™ Reagent Powder. The samples were heated at 55° C. for 10 mins. prior to analysis. Chromatography was performed on an AGILENT (Palo Alto, Calif.) 1100 series HPLC using a WATERS ACCUTAG Amino Acid Analysis Column at a flow rate of 1.0 ml/minute. The fluorescence was monitored at excitation and emission wavelengths of 250 nm and 395 nm, respectively. Sample injection volume was 5.0 microliters.

Table 4 presents the analytical results in terms of nmoles of individual residues detected. The removal of low level contaminants accomplished by the hydroxyapatite chromatography step is striking in that following chromatography all traces of contaminants are absent in the polished lots. The concentration by AAA was determined using the nmol of glutamic acid and the RU Mw of the polymer. In all cases, it is somewhat lower than that determined by HPSEC/MALLS. In all samples the Glx peak was off-scale relative to the other residues and so the quantitative value was extrapolated.

TABLE 4
Quantitative amino acid analysis of Oak capsule lots.
Data is from 20 hr. hydrolyses.

| Residue | 1 | 2 | 3 | 4 |
|---|---|---|---|---|
| Asx^{a} | 17.0 (3.2)^{c} | 17.6 (3.4) | | |
| Ser | 5.7 (1.1) | 5.2 (1.0) | | |
| Glx^{b} | 409 (76.4) | 401.2 (77.3) | 424.6 (100) | 499.6 (100) |
| Gly | 35.5 (6.6) | 32.9 (6.3) | | |
| His | 1.5 (0.3) | 1.5 (0.3) | | |
| Arg | 6.5 (1.2) | 6.0 (1.2) | | |
| Thr | 5.3 (1.0) | 4.8 (0.9) | | |
| Ala | 12.9 (2.4) | 11.7 (2.2) | | |
| Pro | 9.4 (1.8) | 7.9 (1.5) | | |
| Tyr | 2.6 (0.5) | 2.3 (0.4) | | |
| Val | 6.6 (1.2) | 5.5 (1.1) | | |
| Met | 1.5 (0.3) | 1.4 (0.3) | | |
| Lys | 10.3 (1.9) | 10.2 (2.0) | | |
| Ile | 3.4 (0.6) | 3.1 (0.6) | | |
| Leu | 5.3 (1.0) | 4.9 (0.9) | | |
| Phe | 2.7 (0.5) | 2.5 (0.5) | | |
| [PGGA] | 0.524 | 0.514 | 0.544 | 0.640 |
| (mg/mL) | | | | |
| AAA/ | 0.78 | 0.76 | 0.66 | 0.73 |
| HPSEC^{d} | | | | |

^{a}Asx = Asp + Asn
^{b}Glx = Glu + Gln
^{c}Value in parentheses is the percent of total nmol for each residue
^{d}HPSEC concentration data taken from Table 2.

Initial UV-Vis spectroscopy of batch 1 indicated the presence of a broadly absorbing peak in the region of 250 to 290 nm. Por comparison, batch 1 and commercial PAGA (SIGMA, St. Louis, Mo.) were prepared in water at nominal concentrations of 1 mg/mL (w/v) and scanned in a 1 cm path length cell using a PERKIN-ELMER (Wellesley, Mass.) LAMBDA 45 spectrophotometer. The peak maxima at approximately 260 nm for the batch 1 material suggested the presence of contaminating nucleic acid. Agarose gel electrophoresis was performed under standard conditions using a 1% agarose gel with detection by ethidium bromide staining.

Preliminary visualization of the agarose gel midway through the run revealed staining in both lanes in which capsule had been loaded, but upon visualization at the completion of the run, only the lane containing the higher loading of material showed staining. The band was fairly diffuse, with a clear trailing toward the higher molecular weight region of the gel. It was believed that this may be the result of non-ideal migration in the presence of high levels of polyglutamic acid. An initial quantitation using the PICOGREEN fluorescent dye-binding assay (MOLECULAR PROBES, Eugene, Oreg.) yielded a value of 168 ng nucleic acidlmg PGGA.

All four batches were quantitated by PICOGREEN using both DNA (calf thymus, SIGMA, St. Louis, Mo.) and RNA (total yeast, AMBION, Austin, Tex.) as a nucleic acid standard. Prior to analysis of batches, control experiments were run using both standards mixed with varying levels of commercial PAGA to test for interference from the polypeptide. The background response of PAGA alone in the assay was low, and addition of PAGA at 0.5 mg/mL to the standard dilution series did not appreciably decrease the light unit response. It was observed that the total response at a given dilution of nucleic acid was lower for RNA as compared to DNA, which was expected based on the manufacturer's claim of specificity for dsDNA. Table 5 gives the concentrations and weight percentages determined for the capsule lots using both DNA and RNA as the standard.

TABLE 5
Nucleic acid analysis of PGGA capsule lots using both
DNA and RNA for standard curve generation.

| | |
|---|---|
| [nucleic acid] (ng/mL) | wt. % nucleic acid^{a} |

| Batch | DNA | RNA | DNA | RNA |
|---|---|---|---|---|
| 1 | 56 | 257 | 0.00084 | 0.00384 |
| 2 | 97 | 468 | 0.00143 | 0.00688 |
| 3 | 7 | 28 | 0.00008 | 0.00034 |
| 4 | ND^{b} | 6 | ND | 0.00007 |

^{a}Concentration of PGGA as determined by RI from Table 2.
^{b}ND: none detected

It is readily apparent that despite the choice of standard, the residual nucleic acid is very low even in the crude lots. The discrepancy between these results and A260 data, could be explained by either very low molecular weight nucleotides (which are not detected by fluorescence assays) or non-nucleic acid contaminants absorbing at 260 nm.

### EXAMPLE 3

Activation and Conjugation of PGGA and Carrier Protein

This Example presents the details of activation and conjugation, and summarizes the analytical characterization of intermediates and conjugate product.

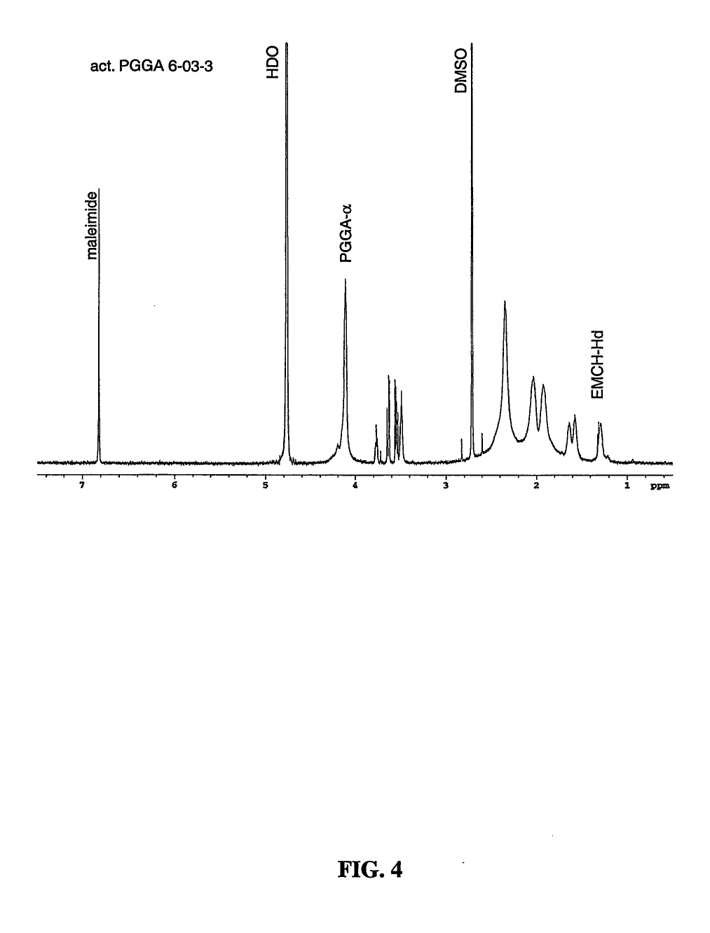

PGGA (30 mg) was converted from the Na+ to TBA+ form by passage through a column of AG50WX8 resin (BIO-RAD, Hercules, Calif.) which had been equilibrated in 1 M tetrabutyl ammonium hydroxide (ALDRICH, St. Louis, Mo.)and exhaustively washed with water. The polymer was dried out of water and 25 mg was dissolved in anhydrous DMF (ALDRICH, St. Louis, Mo.) at 5 mg/mL. Activation was performed by adding EMCH (PIERCE, Rockford, Ill.) at a 1:1 molar ratio to carboxyl followed by DMTMM (ACROS, Geel, Belgium) at a 0.2:1 molar ratio to carboxyl. The reaction vessel was purged with N2 and activation was allowed to proceed for 1 hr on ice followed by 2 hr at ambient T, in the dark. The reaction was diluted 5-fold with water, dialyzed against N2-purged 1 M NaCl, and then exhaustively against N2-purged water in the dark at 2-8° C. The recovered product was concentrated approximately 4fold using a 30 kDa centrifugal concentrator and the recovered bulk was sterile-filtered using an 0.22 micrometer membrane. An aliquot of product was dried and the remainder of the aqueous bulk was stored at −70° C. Maleimide incorporation was estimated by NMR and thiol-consumption assay, and Mw was determined by HPSEC/MALLS. FIG. 4 shows the NMR spectrum and Table 6 summarizes the analytical data.

TABLE 6
Analytical characterization of maPGGA

SCL (%)

| | NMR | thiol-consumption | Mw (Da) |
|---|---|---|---|
| activated PGGA | 10.2 | 7.9 | 522,000 |

Purified sterile OMPC was reacted under aseptic conditions with N-acetylhomocysteine thiolactone to convert a portion of the carrier's lysine residues to thiol groups as generally known in the art (Marburg, S. et al (1986) J. Am. Chem. Soc. 108, 5282-5287; Leanza, W. J., et al, (1992) Bioconjugate Chem. 3, 514-518). Following reagent removal, the thiol content was determined by Ellman's assay and protein content by a modified Lowry assay.

Thawed activated PGGA was buffered to 20 mM HEPES, pH 7.3, and mixed with thiolated OMPC. The final reaction was buffered to 20 mM HEPES, pH 7.3, 0.5 M NaCl, 2 mM EDTA. A thiolated OMPC-only control was carried through in parallel. Conjugation proceeded at ambient temperature for 27 hr in dark. Residual thiols were quenched using a 5-fold molar excess of iodoacetamide for 21 hr, and then residual maleimides were quenched by adding N-acetylcysteanine (ALDRICH, St. Louis, Mo.) at a 5-fold molar excess over iodoacetamide and reacting for 12 hrs.

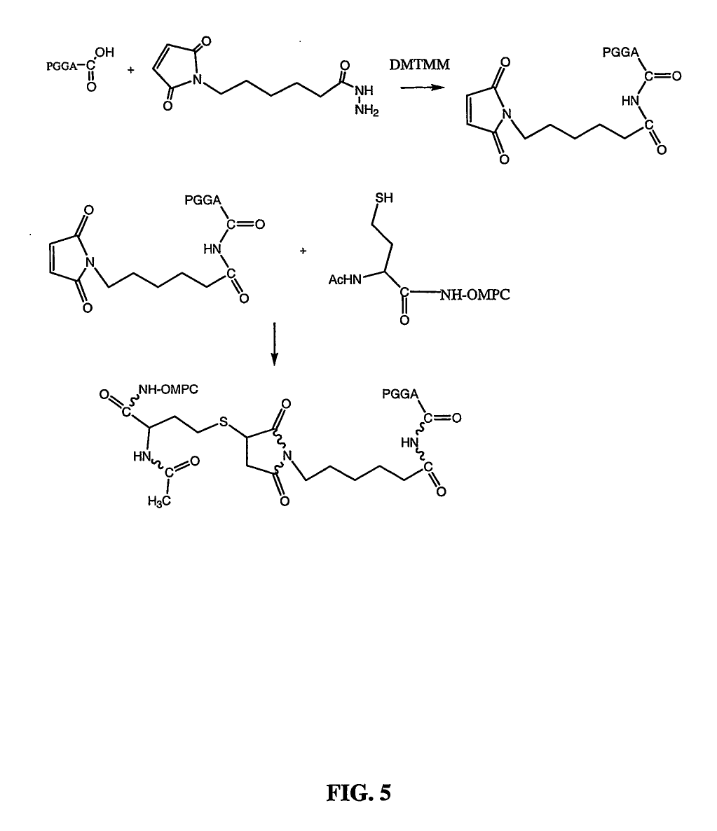

Once reactions were complete, the OMPC-only control was divided in two portions. To one portion was added native non-activated PGGA at the same weight ratio as that present in the conjugate reaction to prepare a physical mixing control. The second portion was untreated. All reactions were incubated for one hour at ambient temperature in dark and were subsequently processed to remove reagents and residual free PGGA by a series of pelleting/resuspension cycles. Briefly, conjugates and controls were centrifuged at 289,000×g for 1 hr. at 4° C. to pellet conjugate. Supernatant was discarded and the pellet was resuspended in 3.0 mL sterile HBS-EDTA (20 mM HEPES, pH 7.3, 0.15 M NaCl, 2 mM EDTA). The resuspended pellet was transferred to a Dounce homogenizer and processed with 30 strokes. The original tube was washed with 2.0 mL HBS-EDTA, the wash processed in a Dounce homogenizer with ten strokes, and the wash combined with resuspended pellet. The pelleting/resuspension was repeated twice, and the final pellet resuspension was in sterile 0.15 M NaCl at a nominal concentration of 3 mg/mL. The resuspended pellet was centrifuged at 1,000×g for 5 min at 40° C., and the supernatant recovered as final product and finally resuspended in sterile saline. Aliquots were taken for analysis and the sterile bulks were stored at 2-8° C. The PGGA activation and conjugation schemes are presented graphically in FIG. 5.

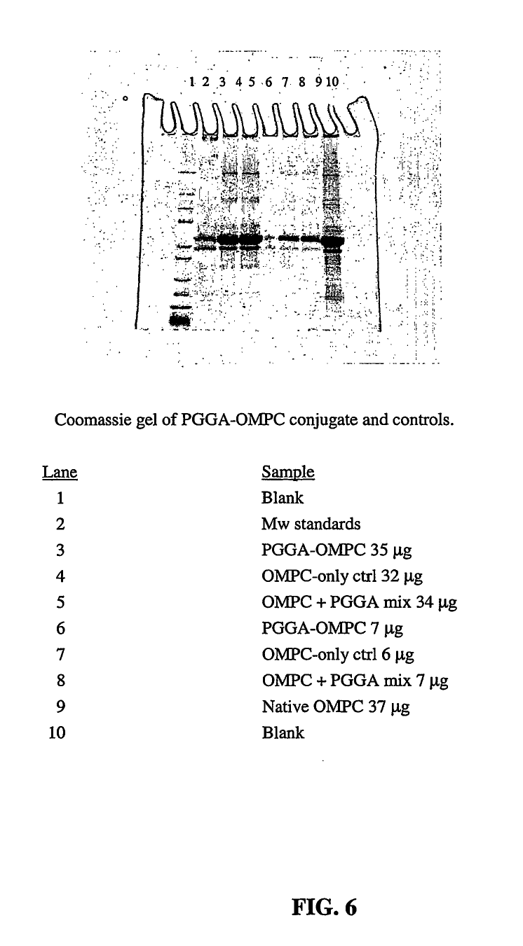

Qualitative evidence for the covalency of the conjugated product was obtained by SDS-PAGE analysis using colloidal Coomassie staining as shown in FIG. 6. All samples were analyzed at two loadings along with a native OMPC control. The major protein species in OMPC migrates at approximately 42,000 as seen for the native and thiolated OMPC-only control. An identical banding pattern is observed for the physical mixing control, indicating no reaction occurs between thiolated carrier and native PGGA. However, the intensity of the monomer band is greatly reduced in the conjugate sample, and minor higher mass components observed in the controls are absent. This demonstrates that covalent attachment of PGGA has proceeded since the high mass of the capsule polymer would effectively prevent migration of the derivatized OMPC monomers into the gel.

Total protein was determined by the modified Lowry assay and samples were subjected to quantitative amino acid analysis (AAA). To insure complete carrier hydrolysis, samples were hydrolyzed in 6N HCl/2% phenol at 110° C. for 70 hrs., dried, resuspended in 20 mM HCI, and analyzed on a WATERS ACCUTAG system after derivatization with WATERS ACCQFLUOR reagent. Samples were heated at 55° C for 10 mins. prior to chromatography on an Agilent 1100 series HPLC using a WATERS ACCUTAG Amino Acid Analysis Column at a flow rate of 1.0 mL/minute with fluorescence detection.

The total OMPC protein was independently calculated from the AAA using compositional data for nine stable residues (excepting Glu) and previously determined nmol residue/mg Lowry protein data generated for native OMPC. The results are shown in Table 7. From this data the expected nmol of Glu for each sample was calculated and compared with the observed as shown in Table 8. Since the OMPC-only control and the physical mixing control gave identical observed:expected values, it was concluded that no non-covalent association of PGGA and OMPC was occurring, and thus the PGGA content of the conjugate was represented by the amount of excess Glu. The unique residue 6-aminohexanoic acid (Aha) was formed by hydrolysis of the incorporated EMCH cross-linker. While it is indicative of the presence of activated PGGA, it is not indicative of covalency. The unique residue formed by hydrolysis of the new covalent bond, dicarboxyethyl homocysteine, was not quantifiable using these analysis conditions. However, Aha was detected only in the conjugate, and comparing the molar ratios of Aha to PGGA, yielded 8.6%, a value which is very close to the 10.2% side chain loading determined by NMR for the activated PGGA prior to conjugation.

TABLE 7
Concentration data for PGGA-OMPC and OMPC-only control

| | Lowry protein | AAA protein | Concentration |
|---|---|---|---|
| Sample | (mg/mL) | (mg/mL) | PGGA (mg/mL) |
| PGGA-OMPC | 2.79 | 2.32 | 0.198 |
| (conjugate) | | | |
| OMPC control | 2.52 | 1.58 | ND |

ND: none detected

TABLE 8
Calculation of PGGA content from AAA data

| | | OMPC- | OMPC + |
|---|---|---|---|
| | PGGA-OMPC | only control | PGGA mix |
| [protein] by AAA (mg/mL) | 2.32 ± 0.26 | 1.58 ± 0.16 | 1.69 ± 0.17 |
| Expected Glu (nmol)¹ | 150.6 | 102.6 | 109.7 |
| Observed Glu (nmol) | 320.6 | 112.6 | 120.7 |
| Observed/Expected | 2.13 | 1.10 | 1.10 |
| Excess Glu (nmol) | 154.9 | 10.0 | 11.0 |
| [PGGA] (mg/mL)² | 0.198 | 0 | 0 |

¹Calculated using 0.6493 nmol Glu/mg protein, determined by average of 9 independent analyses of the starting OMPC lot.
²Excess Glu was for a volume of 0.100 mL or 1,549 nmol Glu/mL. Since PGGA RU = 128.1 and [Glu] = [RU], [RU] = 0.198 mg/mL = [PGGA]. No correction was made for the slight amount of “excess” Glu found in the controls since the ratio was identical for OMPC-only and physical mix samples. For these samples, the discrepancy between observed and expected is within the error range of the analysis (+15%).

The activation and conjugation strategies described herein resulted in a covalent attachment of high Mw PGGA to OMPC carrier protein. The conjugate was fully soluble under physiological conditions.

### EXAMPLE 4

Assay of anti-PGGA Antibody Production

Antibodies against PGGA were measured using the following enzyme immunoassay (EIA). A 96 well COSTAR high binding ELISA plate was coated with 2 μg/ml purified poly-D-glutamic acid capsule, 100 μl/well in PBS and incubated overnight at 4° C. (The antigen-coated plates can generally be stored for up to two weeks prior to use.) The plate was washed 3 times with PBS (plate washer) and blocked with 150 μl/well 5% FBS+PBS+0.1% sodium azide at room temperature for 24 hrs. The plate was washed 3 times with PBS and then 50 μl 5% FBS+PBS+0.1% sodium azide (blocking solution) was added to each well of the plates. Sera was pre-diluted with blocking solution and then 12.5 μl of undiluted or pre-diluted serum was added to each well of row “A”. A 5-fold serial dilution was performed well to well and the plates were incubated at 4° C. overnight and then washed 3 times with PBST (PBS+0.005% Tween-20). The second antibody-horseradish peroxidase (HRP) conjugate[goat anti-mouse (GAM) IgG-HRP, γ-chain specific] SOUTHERN BIOTECH (Birmingham, Ala.) cat #1030-05, diluted 1:6000, was added, 50 ul/well prepared in 5% FBS+PBS. The plates were incubated at room temperature for 2 hrs. and washed 3 times with PBST and 3 times with PBS. Substrate (IMMUNOPURE TMB substrate kit, PIERCE (Rockford, Ill.), cat #34021) was added for 15 minutes. The reactions were stopped with 1M (2N)H₂SO₄ 50μl/well and the plates were read at OD 450 nm.

### EXAMPLE 5

Immunogenicity Experiments

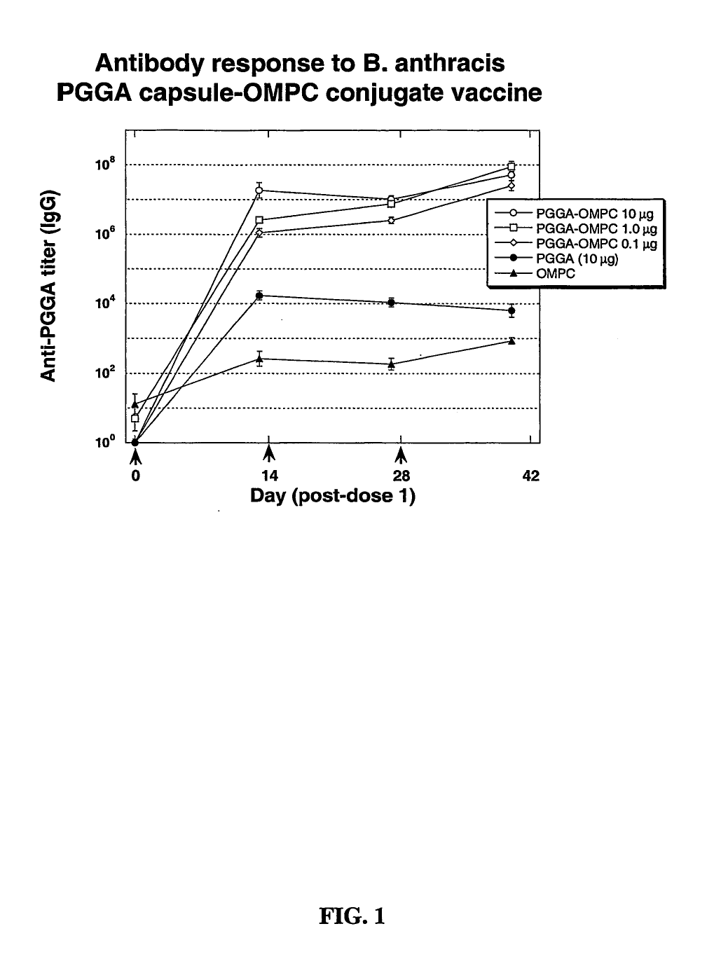

The results of a dose-ranging study in which BALB/c mice were immunized with three dose levels of the PGGA-OMPC conjugates are shown in FIG. 1. Groups of 9-10 mice were injected by the intraperitoneal route with PGGA-OMPC conjugate vaccines containing 10, 1.0 or 0.1 μg doses vaccine (based on the PGGA content of the conjugate) or with unconjugated PGGA (closed circles) or OMPC alone (closed triangles). All components were formulated with Merck aluminum hydroxyphosphate adjuvant.

For this experiment, mice were injected at day 0, 14, 28, and sera were collected on days 0, 14, 28 (prior to vaccination) and on day 40. Sera were tested for IgG antibody titers using an enzyme immunoassay (EIA) using plates coated with PGGA as described above. The results are expressed as the geometric mean endpoint titers (1/dilution) from serum samples assayed in duplicate with the standard errors as indicated. As shown in FIG. 1, mice immunized with the PGGA-OMPC vaccine developed IgG antibody titers (reciprocal of dilution) of greater than 10⁶ just 2 weeks after the first injection. Titers were relatively flat two weeks after the second injection but rose somewhat two weeks following the third injection. In contrast, titers elicited with a 10 μg dose of the unconjugated PGGA were ˜1000 fold lower than the response to a 10 μg dose of the PGGA-OMPC conjugate vaccine. There was some indication of a dose-response to the conjugate vaccine in that the 10 μg dose of PGGA-OMPC induced an approximately 10-fold higher response than did the 0.1 μg dose of the PGGA-OMPC vaccine. The response to the 1.0 μg dose was intermediate between the high and low doses. Differences in response to the high and low dose levels of vaccine were minimal after the third injection at which time titers were higher than 10⁷.

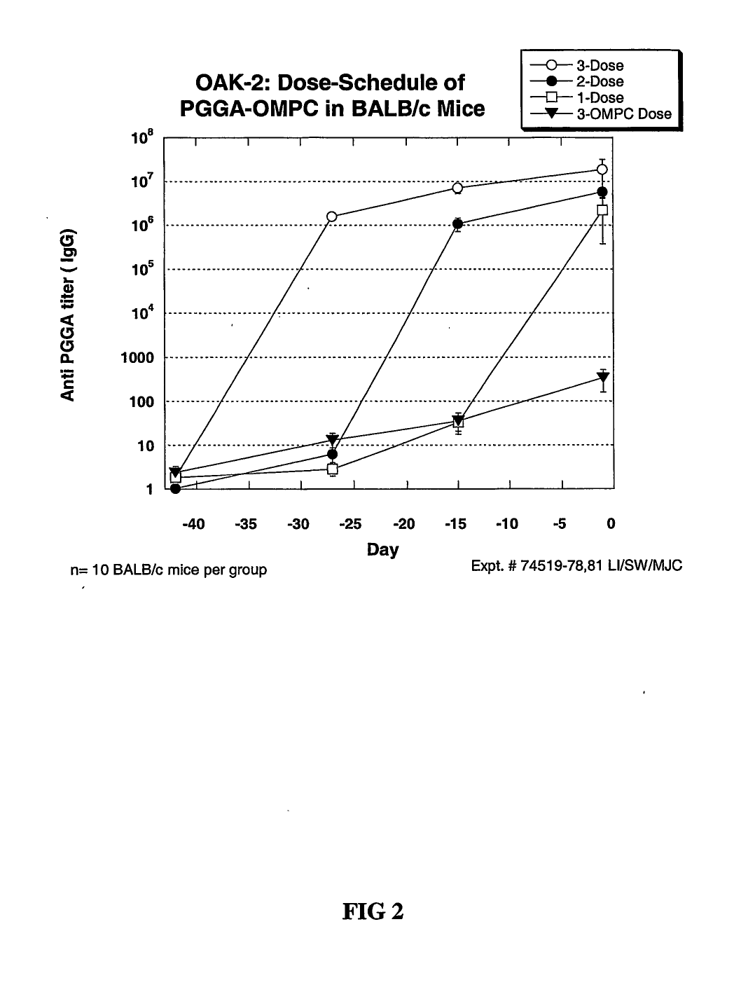

A second immunization and challenge experiment was conducted in groups of 7-10 BALB/c mice to evaluate 3 different dosing schedules for the PGGA-OMPC vaccine formulated with an aluminum hydroxyphosphate adjuvant and administered by the intraperitoneal route of injection. For this experiment, mice in group one were injected on day −42 and day −28 with a 1 mcg dose (based on PGGA content) of the conjugate vaccine and with a 4 mcg dose of the conjugate vaccine on day −14. Mice in group two were injected on day −28 with a 1 mcg dose and on day −14 with a 4 mcg dose of the conjugate vaccine. Mice in group 3 were injected with a single 4 mcg dose on the conjugate vaccine on day −14. Mice in group 4 were injected with OMPC adsorbed to aluminum hydroxyphosphate adjuvant on day −42, day −28 and day −14. Serum from each mouse was collected prior to each injection and on day −2 and tested for anti-PGGA antibody titers. As shown in FIG. 2, mice in each group that received the PGGA-OMPC conjugate vaccine had anti-PGGA IgG titers>10⁶ after a single dose. Mice that received more than one dose had titers of ˜10⁷ by day −2.

### EXAMPLE 6

Efficacy of the Conjugate Vaccine

In the efficacy experiment, mice vaccinated in the dose ranging study (FIG. 1) were challenged by the intraperitoneal injection of ˜1000 colony forming units (cfu) of live virulent B. anthracis (Ames strain). At day 12 post-challenge, only 3/10 mice immunized with free PGGA (group 1) survived whereas all (27/27) of the mice receiving PGGA-OMPC conjugate vaccine (groups 2-4) at dose levels of 10, 1.0, or 0.1 μg per dose survived. In group 5 (OMPC), 4/9 mice survived, and in group 6, 0/5 mice survived. The results (Table 9.) indicate that the PGGA-OMPC vaccine protected 100% of mice, including those receiving the lowest dose tested.

TABLE 9
Dose-ranging of PGGA-OMPC in BALB/c mice Challenge-1

| | | (capsule) Dose | Challenge | |
|---|---|---|---|---|
| Group | Vaccine | (0.5 mL) | (d.42) | #Dead/Total |
| 1 | PGGA | 10 μg | ˜1000 cfu | 7/10 |
| 2 | PGGA-OMPC | 10 μg | ˜1000 cfu | 0/9 |
| 3 | | 1 μg | ˜1000 cfu | 0/9 |
| 4 | | 0.1 μg | ˜1000 cfu | 0/9 |
| 5 | OMPC/Alum | — | ˜1000 cfu | 5/9 |
| 6 | none | none | ˜1000 cfu | 5/5 |

The surviving mice from the first challenge experiment were subsequently re-challenged on day 12 with a higher number (˜5,000 cfu) of virulent vegetative B. anthracis (Ames strain). Within 3 days of challenge, the 4 surviving mice in group 5 (OMPC/alum) were dead. One of the three remaining mice in group 1 (PGGA) died 13 days after re-challenge. By contrast only 2 of the 27 mice in groups 2-4 (PGGA-OMPC conjugate vaccine) died after re-challenge (1 mouse in group 3 died 10 days after re-challenge and 1 mouse in group 4 died 13 days after re-challenge).

Mice from the second immunization experiment (FIG. 2) were challenged at day +7 with 5,000 cfu of vegetative B. anthracis (Ames strain). Within 3 days, all 20 mice in groups 4 and 5 (controls) were dead. In contrast, all mice receiving either 1, 2, or 3 doses of the PGGA-OMPC vaccine survived for the duration of the experiment (21 days). These results (Table 10.) indicate that the PGGA-OMPC conjugate vaccine elicits protection after a single dose.

TABLE 10
Dose schedule experiment in BALB/c mice: Challenge results

| | | | | | Challenge |
|---|---|---|---|---|---|
| | | | | Day +7 | results |
| Group | Day −42 | Day −28 | Day −14 | (i.p. challenge) | #dead/total |
| 1 | PGGA- | PGGA-OMPC | PGGA-OMPC | ˜5000 cfu | 0/8 |
| | OMPC | | | | |
| 2 | | PGGA-OMPC | PGGA-OMPC | ˜5000 cfu | 0/9 |
| 3 | | | PGGA-OMPC | ˜5000 cfu | 0/7 |
| 4 | OMPC-alum | OMPC-alum | OMPC-alum | ˜5000 cfu | 10/10 |
| 5 | none | none | none | ˜5000 cfu | 10/10 |

### EXAMPLE 7

Preparation of Immunogenic Compositions

PGGA conjugate is formulated according to known methods, such as by the admixture of pharmaceutically acceptable carriers, stabilizers, or a vaccine adjuvant. The immunogenic conjugate of the present invention may be prepared for vaccine use by combining with a physiologically acceptable composition such as, e.g. PBS, saline or distilled water. The immunogenic conjugate is administered in a dosage range of about 0.01 to 100 μg, preferably about 1 to about 50 μg or 5 to 25 μg, in order to obtain the desired immunogenic effect. The amount of conjugate per formulation may vary according to a variety of factors, including but not limited to the individual's condition, weight, age and sex. Administration of the conjugate formulation may be by a variety of routes, including but not limited to oral, subcutaneous, topical, mucosal and intramuscular.

An antimicrobial preservative, e.g. thimerosal, optionally may be present. The immunogenic antigens of the present invention may be employed, if desired, in combination with vaccine stabilizers and vaccine adjuvants. Typical stabilizers are specific compounds, e.g. polyanions such as heparin, inositol hexasulfate, sulfated beta-cyclodextrin, less specific excipients, e.g. amino acids, sorbitol, mannitol, xylitol, glycerol, sucrose, dextrose, trebalose, and variations in solution conditions, e.g. neutral pH, high ionic strength (ca. 0.5-2.0M salts), divalent cations (Ca²+, Mg²⁺). Examples of adjuvants are Al(OH)₃, Al(OH)ₓ(SO₄)_{y}(PO₄)_{z} and Al(PO₄). The vaccine of the present invention may be stored under refrigeration or in lyophilized form.

### EXAMPLE 8

Immunogenicity Study in Primates

An immunogenicity study was conducted in primates using PGGA-OMPC vaccine formulated on aluminum hydroxyphosphate adjuvant. Two and one-half micrograms (2.5 μg) of the adjuvanted vaccine was administered to each of three rhesus monkeys on week 0 and week 4. Serum was collected from each monkey prior to vaccination and on weeks 4 and 8 (4 weeks post-dose 2).

Antibodies against PGGA were measured using the following enzyme immunoassay (EIA). COSTAR high binding 96 well ELISA plates were coated with 2 μg/ml purified poly-D-glutamic acid capsule, 50 μl/well in PBS and incubated overnight at 4° C. The plates were washed 3 times with PBS and then blocked with 150 μl/well 5% FBS+PBS+0.1% sodium azide at room temperature for 2-4 hr or at 4° C. overnight. The plates were washed 3 times with PBST (PBS+0.005% Tween-20), and then 50 μl of 5% FBS+PBS+0.1% sodium azide (blocking solution) was added to each well of the plates. Sera was pre-diluted with blocking solution and then 12.5 μl of undiluted or pre-diluted serum was added to each well of row “A”. Five-fold serial dilutions were performed in consecutive wells, and the plates were incubated at 4° C. overnight. After 3 washes with PBST, alkaline phosphatase-conjugated goat anti-rhesus-IgG (H+L) from SOUTHERN BIOTECH (Birmingham, Ala.) cat #6200-04, diluted 1:2000 in blocking solution was added at 50 μl/well. The plates were incubated at room temperature for 2 hr and washed 3 times with PBST and 3 times with PBS. Next, 50 μl/well of the p-nitrophenyl phosphate (p-NPP) enzyme substrate was added and the plate incubated at room temperature for 30 minutes. The reactions were stopped with 3N NaOH, 50 μl/well and the plates were read at OD 405 nm. Endpoint titers were calculated using a cut off of 0.1 OD units.

The results indicate that a substantial antibody response developed by week 4, after only a single injection of vaccine. The titers rose moderately between week 4 and week 8 (4 weeks post-dose 2).

TABLE 11
Immunogenicity of PGGA-OMPC vaccine in Primates.

| | Anti-PGGA IgG response (1/titer) | | |
|---|---|---|---|
| Monkey | Week 0 | Week 4 | Week 8 |
| 1 | 14 | 129,074 | 196,499 |
| 2 | 7 | 23,014 | 38,684 |
| 2 | 2 | 53,077 | 54,313 |

### EXAMPLE 9

Protection against virulent spore challenge in mice immunized with the PGGA-OMPC conjugate vaccine

Three groups of 9 or 10 BALB/c mice were injected intraperitoneally with 0.5 mL volumes of PGGA-OMPC conjugate vaccine formulated to contain 1.0, 0.1, or 0.10 μg per dose (based on PGGA concentration) on day 0 and day 14. All formulations were adsorbed to aluminum hydroxyphosphate adjuvant. A control group of 10 mnice received no injections.

Serum was collected from the vaccinated mice on day 14 and on day 28. Serum IgG antibody titers against PGGA were measured by ELISA as follows: Costar high binding plates were coated with 2 μg/ml (50 μl/well) purified PGGA in PBS and incubated overnight at 4° C. Plates were blocked overnight at 4° C. with 5% FBS. Serum samples were tested at 1:5 serial dilutions on the ELISA plates after they were pre-diluted at 1:10 or 1:100. The plates were then incubated overnight at 4° C. After washing, alkaline phosphatase labeled goat anti-mouse-IgG (SOUTHERN BIOTECH, Birmingham, Ala.) at 1:2000 dilutions was used to detect bound IgG antibody. The plates were developed using p-nitrophenyl phosphate substrate (SIGMA CHEMICAL, St. Louis, Mo.) and absorbances were measured at 405 nm.

Approximately 6 weeks after the day 28 bleed, all four groups of mice were challenged with a subcutaneous injection of ˜19,000 Bacillus anthracis spores (Ames strain). Survival was monitored for 2 weeks. From the data, it is evident that vaccination with even the lowest dose (0.01) μg of the vaccine resulted in good (80%) protection from challenge with spores from a virulent strain of anthrax.

TABLE 12
Protection against virulent spore challenge

| | | | Anti-PGGA | | Spore |
|---|---|---|---|---|---|
| | | | IgG Titer | | Challenge |
| Group | Immunogen | Dose (μg) | Day 14 | Day 28 | (Survivors^{a}) |
| 1 | PGGA-OMPC | 1 | 25,718 | 2,070,645 | 9/9 |
| 2 | PGGA-OMPC | 0.1 | 8,972 | 640,466 | 9/10 |
| 3 | PGGA-OMPC | 0.01 | 1,431 | 62,602 | 8/10 |
| 4 | none | none | n.d. | n.d. | 1/10 |

^{a}Number of survivors 14 days after subcutaneous challenge with ˜19,000 B. anthracis spores (Ames strain)

What is claimed:

1. A conjugate comprising poly-D-gamma glutamic acid covalently linked to an immunogenic carrier protein wherein the poly-D-gamma glutamic acid is above about 100 kDa.

2. A conjugate comprising poly-D-gamma glutamic acid covalently linked to an immunogenic carrier protein wherein the poly-D-gamma glutamic acid is above about 200 kDa.

3. A conjugate comprising poly-D-gamma glutamic acid covalently linked to an immunogenic carrier protein wherein the poly-D-gamma glutamic acid is above about 300 kDa.

4. The conjugate according to any of claims 1-3 wherein the poly-D-gamma-glutamic acid is covalently linked to the carrier protein by N-(epsilon-maleimidocaproic acid)hydrazide.

5. The conjugate according to any of claims 1-3 wherein the carrier protein is selected from the group consisting of outer membrane protein complex (OMPC) of Neiserria meningitides, tetanus toxoid, diphtheria toxoid, Hepatitis B Surface Antigen (HBsAg), Hepatitis B core antigen (HBcAg), recombinant Protective Antigen or the L1 protein of the Human Papilloma Virus Virus Like Particle type 6, 11 or 16.

6. The conjugate according to any of claims 14 wherein the carrier protein is the outer membrane protein complex of Neiserria meningitidis.

7. A vaccine comprising a conjugate of any of claims 1-6, an adjuvant and a pharmaceutically acceptable excipient.

8. A vaccine comprising a conjugate of poly-D-gamma glutamic acid covalently linked to the outer membrane protein complex of Neiserria meningitidis by N-(epsilon-maleimidocaproic acid)hydrazide, an adjuvant and a pharmaceutically acceptable excipient.

9. A vaccine according to any of claims 7 and 8 further comprising at least one antigen selected from the group consisting of from Haemophilus influenza, hepatitis viruses A, B, or C, epitopes derived from the M2, hemaglutinin and neuraminidase proteins of Influenza virus types A or B, human papilloma virus, measles, mumps, rubella, varicella, rotavirus, Streptococcus pneumonia and Staphylococcus aureus.

10. A method of vaccinating a patient comprising administering an effective amount of a vaccine of any of claims 7-9.

11. A method of making a conjugate of poly-D-gamma glutamic acid and a carrier protein comprising activating the poly-D-gamma glutamic acid on a portion of its carboxylic acid side chains under non-aqueous conditions, introducing thiol reactive groups at the activated side chains and reacting the thiol reactive groups with a sulfhydryl containing carrier protein.

12. The method according to claim 11 comprising,
providing purified poly-D-gamma glutamic acid as a hydrogen or tertbutylammonium salt, and
removing water from the salt, and
dissolving the salt in an organic solvent, and
mixing the salt with N-(epsilon-maleimidocaproic acid)hydrazide, and
adding an activating agent selected from the group consisting of N,N′-diisopropyl carbodiimide and 4-(4,6-dimethoxy[1,3,5]triazin-2-yl)4-methyl-morpholinium chloride, and
diluting the reaction, and
dialyzing the reaction; and
adding thiolated outer membrane protein complex, and
quenching residual thiols, and
isolating the conjugate.

13. A method of purifying poly-D-gamma glutamic acid comprising,
dissolve partially purified extract containing poly-D-gamma glutamic acid in water, and
mixing the solution with 0.004M sodium phosphate, pH 7.0+1M NaCl, and
load mixture on hydroxyapatite chromatography column, and
washing out non-bound material with 0.004M sodium phosphate, pH 7.0+1M NaCl, and
eluting poly-D-gamma glutamic acid with a linear gradient from 0 to 100% 0.4M sodium phosphate, pH 7.0+1M NaCl.
